(19) United States
(12) Patent Application Publication
(10) Pub. No.: US 2022/0330927 A1
(43) Pub. Date: Oct. 20, 2022

(54) Disposable Multiple Needle Biopsy Instrument and Needle Guide

(71) Applicants: Michael Mooreville, Lansdowne, PA (US); Michael Gazzaniga, Fullerton, CA (US)

(72) Inventors: Michael Mooreville, Lansdowne, PA (US); Michael Gazzaniga, Fullerton, CA (US)

(21) Appl. No.: 17/591,555

(22) Filed: Feb. 2, 2022

Related U.S. Application Data
(60) Provisional application No. 63/149,609, filed on Feb. 15, 2021.

(51) Int. Cl. A61B 10/02

(52) U.S. Cl. CPC A61B 10/0241

(57) ABSTRACT

Multiple needle biopsy instruments, guides, and systems and methods for forming and using the same are disclosed. In some embodiments, the multiple needle biopsy instrument can include a handle, a first needle and second needle, an inner core platform, an outer sheath platform, and a set of trigger mechanism controls. The first and second needles can include inner cores formed with tissue notches, and outer sheaths. The inner core and outer sheath platforms can be joined to the inner cores and outer sheaths, and coupled to inner core and outer sheath springs. The set of trigger mechanism controls can control movement of the inner core and outer sheath platforms as well as the inner core and outer sheath springs. Releasing the inner core and outer sheath springs causes the outer sheaths and inner core to eject such that tissue cut by the outer sheath is deposited into the tissue notches.

### CROSS REFERENCE TO RELATED APPLICATIONS

This application claims priority to U.S. Provisional Patent Application No. 63/149,609, filed Feb. 15, 2021, which is hereby incorporated by reference herein in its entirety.

### FIELD OF INVENTION

The present invention is related to surgical medical devices used with prostate tissue biopsies, and more particularly, to systems and methods for making and using biopsy needles for removing prostate tissue.

### BACKGROUND OF INVENTION

Organ tissue pathologic diagnosis is currently performed by means of a tissue biopsy. Most often, these biopsies are performed using a biopsy needle, which can remove a thin strip of tissue to be sent for pathologic diagnosis under microscopic magnification.

In the case of prostate tissue, a patient may need to have a large number of biopsies performed in a single operation. For example, the number of biopsies performed on a patient in a single operation can range between twelve (12) to eighteen (18). Each biopsy typically involves jabbing the patient's prostrate to retrieve the desired tissue. As a result, a patient may endure a large number of jabs from a biopsy needle during a single operation. Even though these biopsies are typically performed under local anesthesia, given the large number of jabs patients endure, patients still complain of discomfort during and after the procedure. Moreover, the large number of jabs lengthens the duration of the procedure, because the jabs are typically performed sequentially.

Further, the locations of pathologic nodule placement are generally imprecise. Because pathologic nodule placement is determined with the aid of visualization by an ultrasound (“US”) probe or magnetic resonance imaging (“MRI”) fusion equipment, the location of pathologic nodule placement is generally only approximate and the potential risk of missing the target lesion with the needle is increased.

Accordingly, devices, systems and methods are needed to perform tissue biopsies that reduce the number of jabs inflicted on a patient and reduce the duration of the procedure, while also decreasing the potential of missing the target lesion.

### SUMMARY OF INVENTION

Embodiments for a multiple needle biopsy instrument, and systems and methods for using and making the same are disclosed. In some embodiments, the multiple needle biopsy instrument can include a handle formed with a hollow body, a first needle and second needle, an inner core platform, an outer sheath platform, and a set of trigger mechanism controls.

The first needle can include a first inner core formed with a first tissue notch, and a first outer sheath that is formed coaxially around the first inner core. The second needle can include a second inner core formed with a second tissue notch, and a second outer sheath that is formed coaxially around the second inner core.

The inner core platform can be joined to the first and second inner cores, and coupled to an inner core spring. The inner core platform can move the first and second inner cores and the inner core spring along the hollow body of the handle. The outer sheath platform can be joined to the first and second outer sheaths, and coupled to an outer sheath spring. The outer sheath platform moves the first and second outer sheaths and the outer sheath spring along the hollow body of the handle.

The set of trigger mechanism controls can include an inner core button, an outer core button, and a trigger release button. The inner core button can be configured to control movement of the inner core platform joined to the first and second inner cores. The outer sheath button can be configured to control movement of the outer sheath platform joined to the first and second outer sheaths. The trigger release button can be configured to release the inner core spring and outer sheath spring.

Actuation of the trigger release button causes the outer sheath platform to eject after the inner core platform. The outer sheaths of the first and second needles cut tissue as they extend over the first and second inner cores. As the outer sheaths of the first and second needles continue to extend over the inner cores, they deposit the cut tissue in the tissue notches of the first and second needles.

In some embodiments, a system for collecting tissue can include a multiple needle biopsy instrument and a guide. The guide can include a first surface for supporting a probe, a needle entry barrel, a needle exit barrel, and a needle channel. The first surface can be configured to guide the multiple needle biopsy instrument to a targeted destination. The first surface can also be formed with one or more wings for securing the probe. The needle entry barrel can be formed at the distal end of the guide and configured to receive the first needle and second needle. The needle exit barrel can be formed at the proximal end of the guide and configured to discharge the first needle and second needle. The needle channel can extend from the needle entry barrel to the needle exit barrel. In this way, the guide can be configured to direct the first needle and second needle to the targeted destination.

### BRIEF DESCRIPTION OF THE DRAWINGS

The novel features of the invention are set forth in the appended claims.

However, for purpose of explanation, several embodiments of the invention are set forth in the following figures.

FIGS. 1A, 1B, 1C, 1D, 1E, 1F, 1G, 1H, 1I, 1J, and 1K illustrate perspective views of the multiple-needle biopsy instrument according to embodiments of the invention.

FIGS. 2A, 2B, and 2C depict side views of a guide for multiple-needle biopsy instruments according to embodiments of the invention.

Like reference symbols in the various drawings indicate like elements.

### DETAILED DESCRIPTION OF THE INVENTION

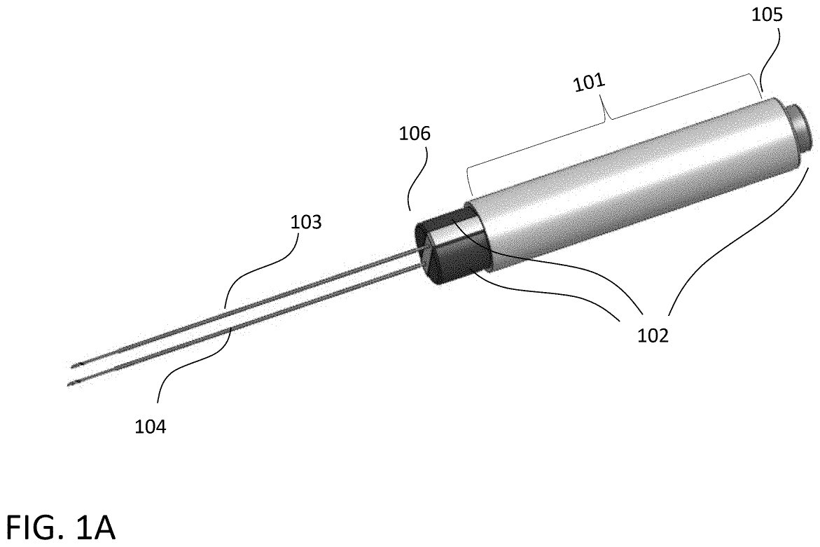

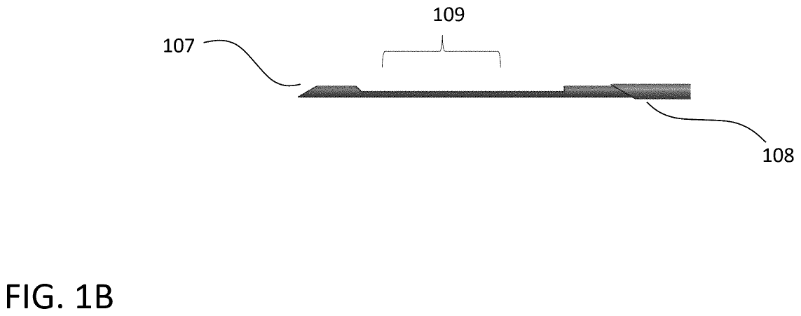

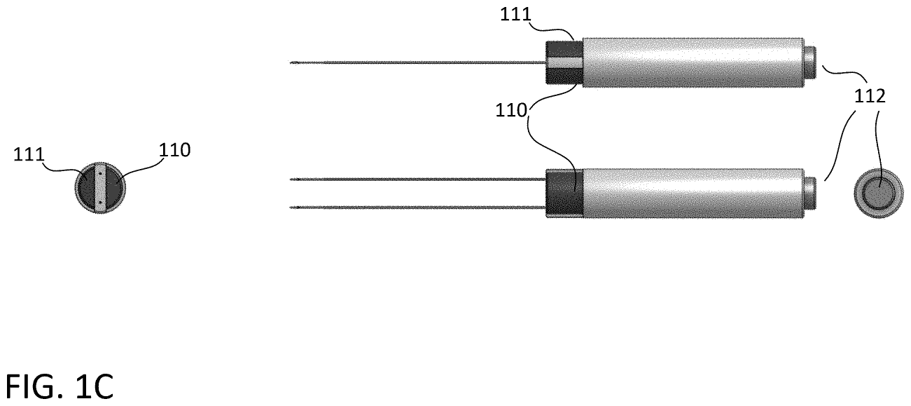

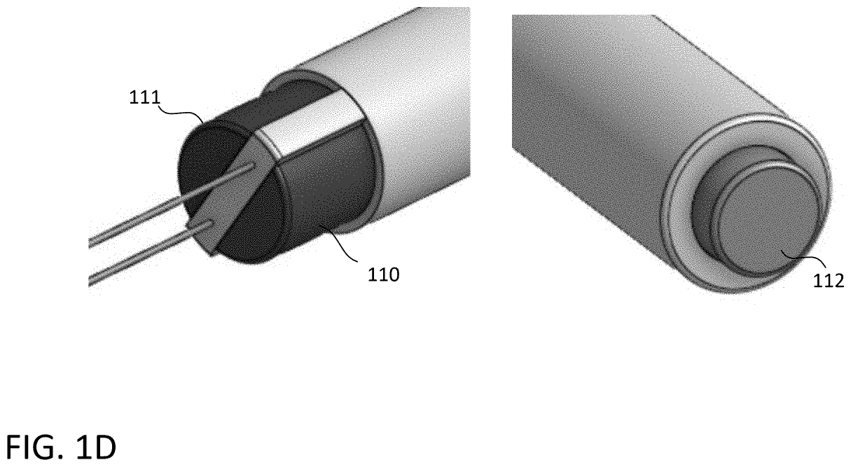

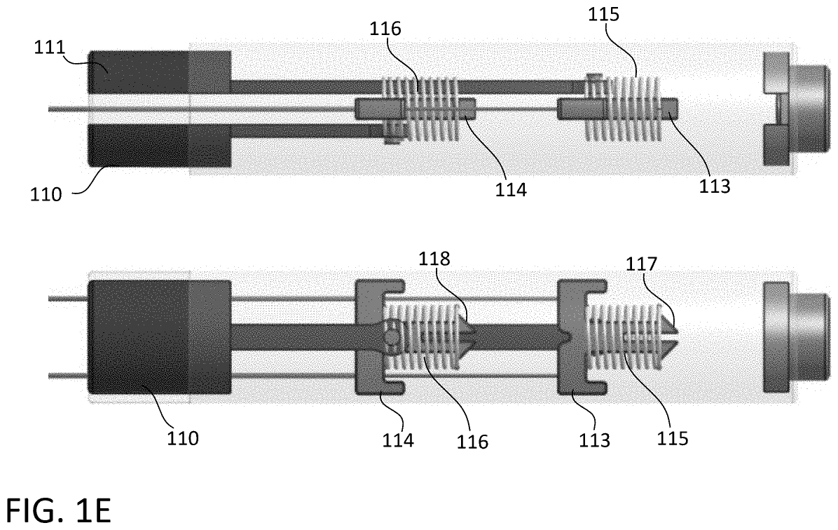

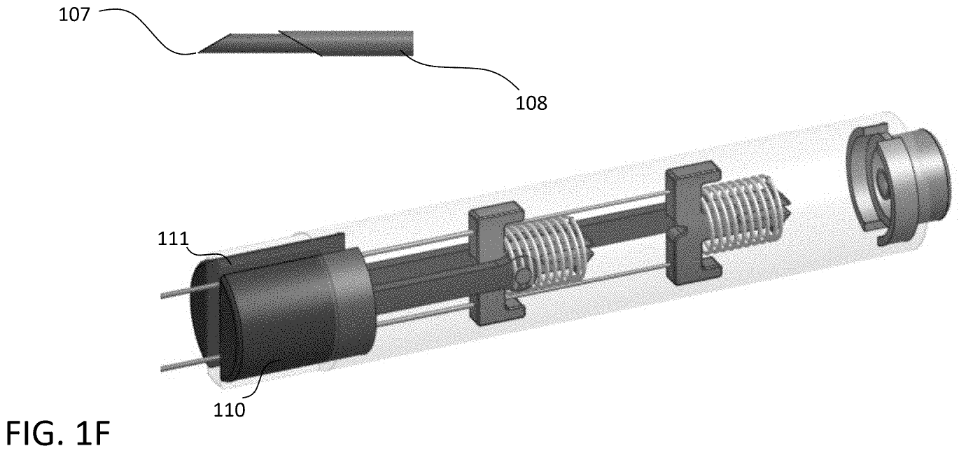

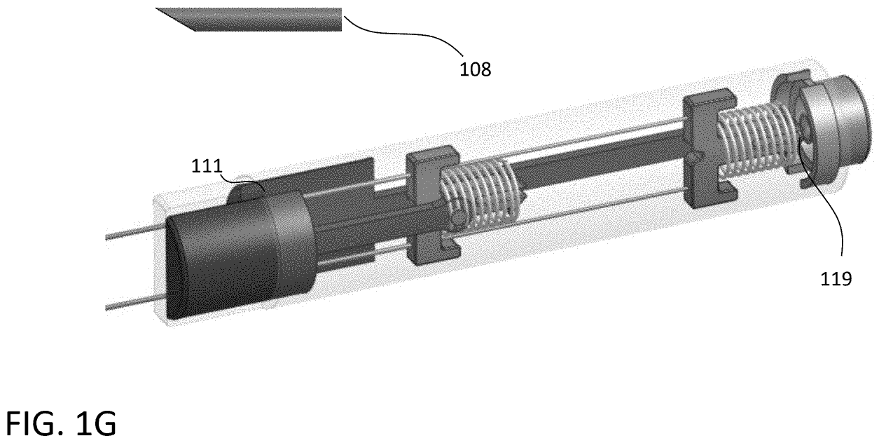

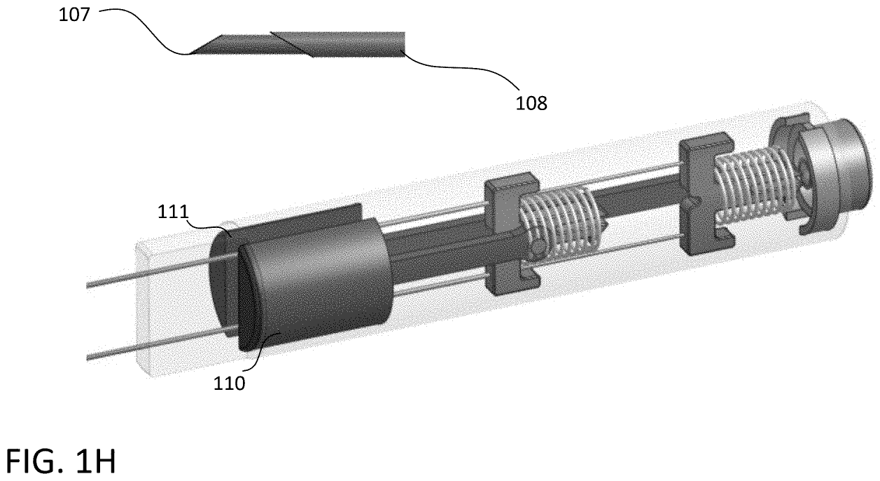

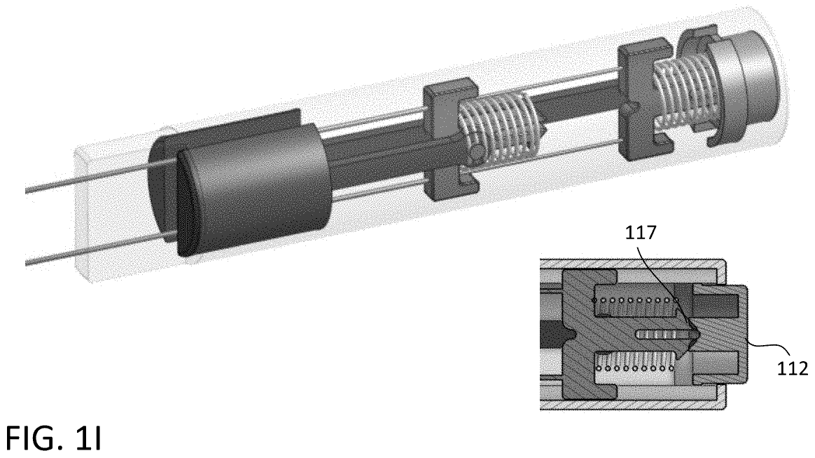

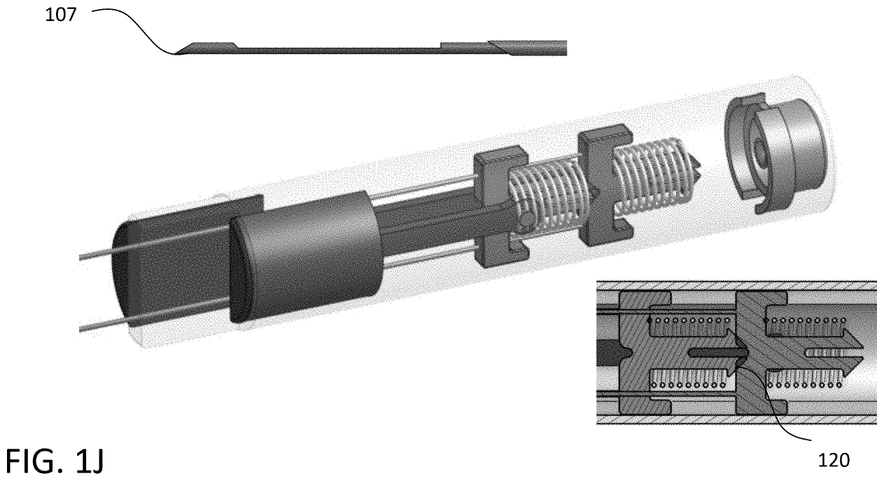

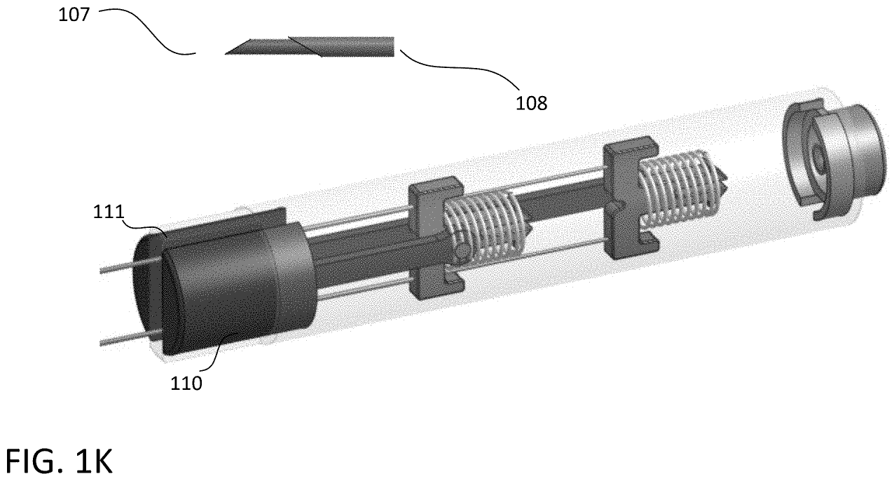

A disposable multiple-needle biopsy instrument according to some embodiments of the invention is shown in FIG. 1A. The disposable multiple-needle biopsy instrument can include a handle 101, a set of trigger mechanism controls 102, a first needle 103, and a second needle 104. In some embodiments, handle 101 has a hollow core for housing the first and second needles and a trigger mechanism. When the first and second needles are partially or completely retracted using the set of trigger mechanism controls, they are housed within the hollow core of the handle until they are ejected using springs as described in more detail below. The set of trigger mechanism controls can be operated to release the first and second needles in a manner that cuts a small strip of tissue to biopsy.

As shown in FIG. 1A, the longitudinal axes of the first and second needles can be formed parallel to the longitudinal axis of the handle extending from the distal end 105 to the proximal end 106. The distance between the centers of the first and second needles can vary depending on the gauge of the needles being used. For example, if the needle gauges are each 14 G they can be formed about 0.8 to 1 cm apart (as measured between their centers). The first and second needles can have gauges of the same or different sizes. For example, a combination of a large and small gauge can be used to collect two samples of different sizes. The first and second needles can also be formed to have different lengths.

According to the embodiments shown in FIG. 1B, the first and second needles can each comprise an inner core 107, and an outer sheath 108. The outer sheath can be formed coaxially around the inner core such that it can glide over the surface of the inner core when ejecting from, or retracting within the hollow core of the handle. The outer sheath can be formed with a sharp cutting edge so that when the outer sheath glides over the inner core, it can cut tissue that it comes into contact with. A tissue notch 109 can be formed at the proximal end of the inner core so that when the sheath cuts tissue, the tissue will collect in the notch.

In some embodiments, the set of trigger mechanism controls can include an outer sheath button 110, an inner core button 111, and a trigger release button 112 as shown in FIG. 1B. Outer sheath button 110 can be configured to control the outer sheaths of the first and second needles such that when the outer sheath button 110 is pressed, the outer sheaths partially retract into the hollow core of the handle. Similarly, inner core button 111 can be configured to control the inner core of the first and second needles such that when the inner core button 111 is pressed, the inner core of the first and second needles retract into the hollow core of the handle.

Inside the hollow core of the handle, the first and second inner cores are joined to an inner core platform 113. The inner core platform is coupled to an inner core spring 115 that compresses and extends from the proximal end of the handle to the distal end of the handle. The inner core button is joined to the inner core platform thereby controlling the movement of the inner core platform and joined first and second inner cores. The inner core button can be joined to the inner core platform with an eyelet hook and peg.

The first and second outer sheathes are joined to an outer sheath platform 114. The outer sheath platform is coupled to an outer sheath spring 116 that compresses and extends from the proximal end of the handle to the distal end of the handle. The outer sheath button is joined to the outer sheath platform thereby controlling the movement of the outer sheath platform and joined first and second outer sheaths. The outer sheath button can be joined to the outer sheath platform with an eyelet hook and peg.

In some embodiments, the inner core platform is positioned at the distal end of the handle and the outer sheath platform is positioned at the proximal end of the handle as shown in FIGS. 1E-1K. The inner core button can join the inner core spring by extending the joining mechanism inside and through opening of the outer sheath spring.

FIG. 1F illustrates the outer sheath and inner core buttons in an unloaded state, before either button has been pressed, and the corresponding position of the inner cores and outer sheaths of the first and second needles according to embodiments of the invention. As shown in FIG. 1G, the inner core button can be pressed towards the distal end of the handle causing the coil of the inner core spring to compress, and the inner cores of the first and second needles to retract inside the outer sheath. As shown in FIG. 1H, the outer sheath button can be pressed towards the distal end of the handle causing the coil of the outer sheath spring to compress, and the outer sheaths of the first and second needles to retract.

The inner core platform and outer sheath platform can be coupled to an inner core locking mechanism 117 and outer sheath locking mechanism 118, respectively. The inner core and outer sheath locking mechanisms can be disposed within the interior of the inner core and outer sheath springs. As the outer sheath and inner core buttons are pressed, the platform can cause the inner core and outer sheath locking mechanisms to advance within the interior of the inner core and outer sheath springs until they reach locking positions. According to some embodiments of the invention, the locking mechanism is substantially conical with one or more flanges at the base of the cone. Once the flanges advance past the last coil of the spring, the flange moves outside the interior of the spring in a locking position. In the locking position, the flange hooks onto the last coil of the spring and keeps the corresponding inner core and/or outer sheath button in place and the inner core and/or outer sheath coil spring compressed until released by the trigger release button. When the outer sheath and inner core buttons are both pressed and reach the locking position, the inner core and outer sheath springs become engaged with the trigger mechanism, thereby loading the multiple-needle biopsy instrument.

With the springs engaged with the inner core and outer sheath of the first and second needles, the trigger release button 112 can be pressed. Inside the hollow core of the handle, the trigger release button is joined to a trigger mechanism that can unlock the locking mechanism and release the coil of the spring from its compressed state. According to some embodiments the trigger release button unlocks the locking mechanism by forcing the flange back into the interior of the spring as shown in FIG. 1I. For example, the trigger mechanism can be a formation 119 disposed on the interior wall of the trigger release button and shaped in a manner that pinches the outer surface of locking mechanism inward. As shown in FIG. 1I, the formation 119 can be a protrusion that is substantially cylindrical such that when it comes into contact with the conical surface of the locking mechanism, the flange contracts inward into the interior of the spring, which in turn, causes the locking mechanism to unlock, freeing the inner core spring coil from its compressed stated. The inner core spring coil can release its stored energy and eject the inner core platform towards the outlet on the proximal end of the handle.

Pressing the trigger release button causes the inner core and outer sheath locking mechanisms to unlock and release the spring from its compressed state. As shown in FIG. 1J, when the inner core platform is positioned closer to the distal end, the trigger release button causes the inner core locking mechanism to unlock first. As the inner core spring decompresses, it ejects the inner core platform towards the outer sheath platform. The ejection of the inner core platform causes forcible contact with the outer sheath platform and, in turn, causes the outer sheath locking mechanism to unlock. The outer sheath spring then decompresses, causing the outer sheath platform to eject. As shown in FIG. 1K both platforms can eject, causing the outer sheaths and inner cores of the first and second needles to advance toward the proximal end of the handle. As the outer sheaths of the first and second needles advance, they cut and deposit tissue into the respective tissue notches of the first and second needles for subsequent collection.

The inner cores of the first and second needles can be ejected first. As shown in FIGS. 1E-1K, when the inner core platform is located at the distal end of the handle, it will be the first to advance when the trigger release is pressed. As the inner core platform advances first, the inner cores of the first and second needles extend from the outer sheaths of the first and second needles until the tissue notches of the first and second needles are exposed. The inner core platform continues to advance towards the proximal end of the handle until it contacts the outer sheath platform as shown in FIG. 1J. The inner core platform can be formed with a divot 120 that disengages the outer sheath locking mechanism. The divot can be formed such that when it exerts force on the flange of the outer sheath locking mechanism, the flange is contracted radially inward unlocking the locking mechanism and freeing the outer sheath spring coil from its compressed stated. The outer sheath spring coil can release its stored energy and eject the outer sheath platform towards the outlet of the handle. In this way, the trigger release button can sequentially disengage the lock of the inner core spring before the lock of the outer sheath spring is disengaged. This can allow the tissue notches of the inner core to be exposed and collect tissue from the outer sheaths once they are ejected.

As the outer sheaths of the first and second needles glide over the inner cores of the first and second needles, the sharp edges of the outer sheaths cut and push the tissue it comes into contact with. As the outer sheaths continue to glide over the inner cores, the outer sheaths push the tissue samples it has cut into the tissue notches of the inner cores.

In some embodiments, the coil of the inner core spring can have a different tension strength than the coil of the outer sheath spring. This can be to support needles of different gauges or sizes, or to support additional needles as described in more detail below. The different tension strengths can also be used to vary the speed or timing with which the inner cores and outer sheaths are ejected.

The outer sheath platform can continue to advance proximally towards the outlet of the handle until the energy of the outer sheath spring has fully dissipated as shown in FIG. 1K. As shown in FIG. 1K, in this state, the outer sheaths enclose the tissue notches of the first and second needle. To collect the tissue from the tissue notch, the outer sheath button can be pressed, thereby retracting the outer sheaths and revealing the tissue inside the tissue notch. A medical practitioner can then easily remove the tissue samples from the tissue notch to perform the biopsy.

In some embodiments, the set of trigger mechanism controls can be formed on the handle so that they can be operated with one hand. For example, the outer sheath button and inner core button on the handle can be situated at the top of the proximal end of the handle so they can be easily accessed by an index finger, while the trigger release button can be placed at the distal end of the handle. In further embodiments, the trigger release button can be formed on the side of the handle so it can be easily accessed by a thumb. By situating the trigger mechanism controls in this manner, the multiple-needle biopsy instrument can be manipulated in one hand, allowing a medical practitioner free use of his or her second hand.

In some embodiments, the multiple needle biopsy instrument can be configurable to allow for additional needles to be used to further reduce the number of jabs perceived by the patient and to reduce the overall duration of the procedure. For example, the first and second needles can be detachably joined to an adaptable needle mount for supporting multiple needle configurations. The adaptable needle mount can have a joining mechanism such as a plug or female receptacle on the proximal facing surface of the inner core and outer sheath platforms. The first and second needles can be formed on a matching surface that can detachably join with the plug or female receptable. In this way, the first and second needles can be removed and substituted with a new configuration of multiple needles. For example, the adaptable needle mount can support a new configuration having three needles, where the first and second needles are as described above, and the third needle has a third outer sheath, a third inner core, and a third tissue notch in the same manner as described above. With three needles, the number of jabs perceived by the patient is reduced, as well as the overall duration of the procedure. Even more needles can be added based on the gauge of needles being used, the size of the inner core and outer sheath platforms, and the desired distance between needles. The adaptable needle mount can also be used to support different spatial arrangements of needles. For example, one set of first and second needles having a distance of 0.8 cm apart can be detached and substituted with a different set of first and second needles having a greater distance, such as 1 cm, apart. As more needles are added, springs with greater strength can be used.

In some embodiments, the multiple-needle biopsy instrument is made of disposable medical grade materials that can be discarded after use for a single patient. For example, the needles can be constructed of surgical steel and the handle can be constructed of medical grade plastic.

While the multiple-needle biopsy instrument is disposable, it can still be reused multiple times during a single operation. For example, after a first collection of tissue using the multiple-needle biopsy instrument, the outer sheathes and inner cores can be retracted and reloaded for further tissue collection.

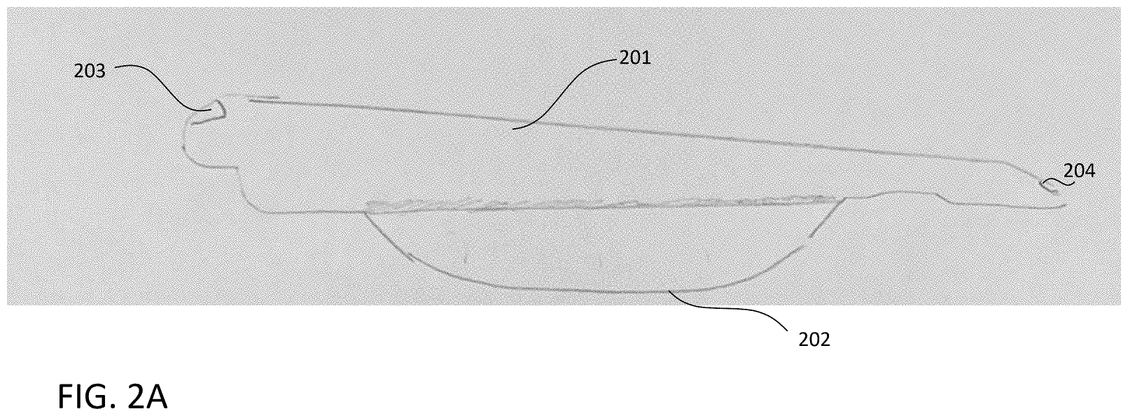

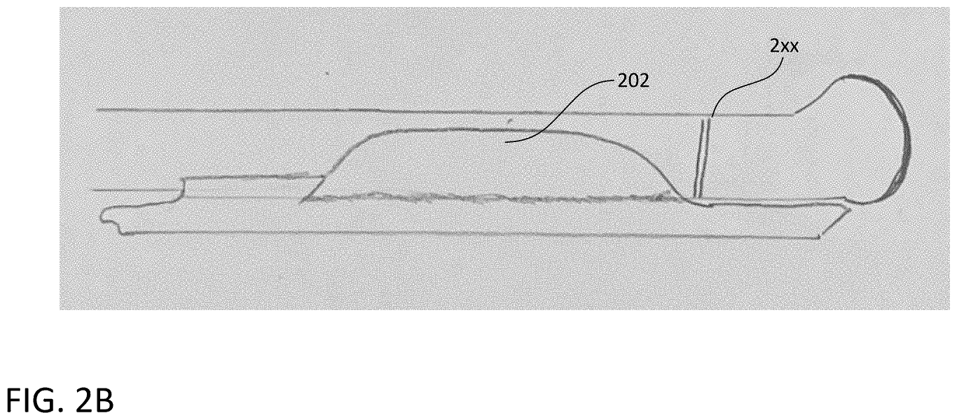

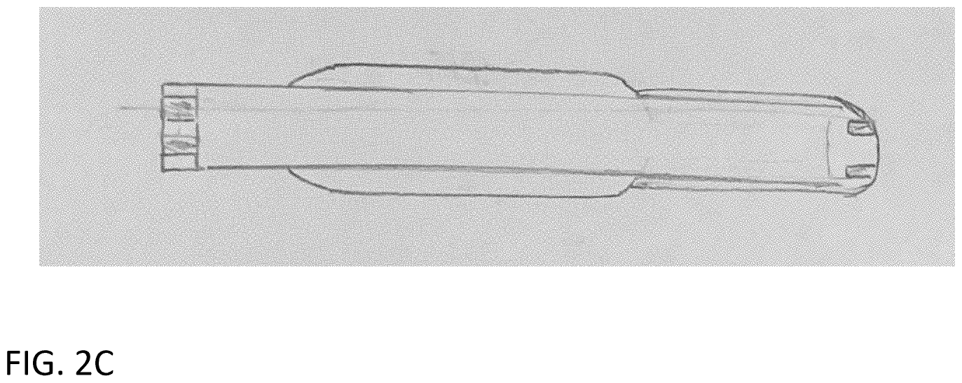

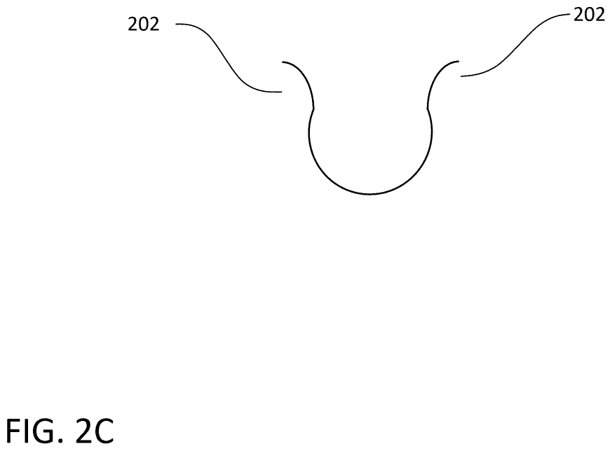

A guide for the multiple-needle biopsy instrument according to some embodiments of the invention is shown in FIGS. 2A-2C. The guide can include a first surface 201 for supporting a probe that is used to guide the multiple-needle biopsy instrument to its destination, one or more wings 202 for securing the probe in place within the guide, a needle exit barrel 203 formed at the proximal end of the guide, a needle entry barrel 204 formed at the distal end of the guide, and a needle channel 205 formed between the needle exit barrel and needle entry barrel.

The first surface can be formed to substantially enclose the probe such that when the probe is inserted into the enclosure it secures the probe in place. A probe can be inserted with the probe head placed at the proximal end of the guide. In this way, when the probe head is used to navigate to a location for collecting tissue within the body, the needle exit barrel will be aimed at the desired location of tissue collection. When the medical practitioner is ready to collect a tissue sample, the medical practitioner can insert the needles of the multiple needle biopsy instrument in through the needle entry barrel. The needle channel 205 of the guide will direct the needles to the needle exit barrel so that it can collect tissue at the precise area aimed at by the probe.

The needle entry barrel, needle channel, and needle exit barrel can be formed to match the configuration of the needles on the multiple needle biopsy instrument. For example, if the multiple needle biopsy instrument includes two needles that are spaced horizontally at 0.8 cm apart, the needle entry barrel, needle channel, and needle exit barrel can be formed the same distance and location apart. Similarly, the size of the needle entry barrel, needle channel, and needle exit barrel can be formed with a size to match the first needle and second needle. For example, the diameter of the needle entry barrel, needle channel, and needle exit barrel can be formed to match the gauge of the first needle and second needle. Similarly, the needle entry barrel, needle channel, and needle exit barrel can be formed with a length to match the length of the first needle and second needle.

The first surface can include one or more wings 202 on the longitudinal sides that flare radially away from the center of the enclosure as shown in FIG. 2C. The wings enhance the secure fit of the probe into the guide. A medical practitioner can snap the probe into the enclosure of the guide between a pair of wings. The wings can also serve as a lever for opening the enclosure so that the probe can be removed from the guide.

Once a probe is secured within the guide, a medical practitioner can use the probe to locate the area on the patient for collecting tissue to biopsy. For example, to conduct a trans-rectal biopsy, the guide can be used to direct the probe through the rectum to the appropriate area on the rectal wall where tissue can be retrieved. The guide and probe head can be positioned at the appropriate area for collecting tissue such that the needle exit barrel is directly aimed at the desired tissue. With the needle exit barrel aimed at the desired tissue, the needles of the multiple needle biopsy instrument can be inserted into the matching barrels of the guide. When the needle biopsy instrument is triggered, the guide directs the needles toward the precise location of tissue collection.

First surface 201 can be formed to have a texture or pattern that improves the grip of the guide and the probe that lies inside the guide. For example, the first surface can be formed with ridges or dimples to enhance the grip for the medical practitioner manipulating the probe.

According to some embodiments of the invention, the probe is used as a visual aid to help identify the targeted area for tissue for collection. Thus, the probe can be a US probe or MRI fusion equipment.

What is claimed is:

1. A multiple needle biopsy instrument comprising:
a handle formed with a hollow body;
a first needle comprising a first outer sheath and a first inner core, wherein the first inner core is formed with a first tissue notch and the first outer sheath is formed coaxially around the first inner core;
a second needle comprising a second outer sheath and a second inner core, wherein the second inner core is formed with a second tissue notch and the second outer sheath is formed coaxially around the second inner core;
an inner core platform joined to the first and second inner cores, and coupled to an inner core spring, wherein the inner core platform moves the first and second inner cores and the inner core spring along the hollow body of the handle;
an outer sheath platform joined to the first and second outer sheathes, and coupled to an outer sheath spring, wherein the outer sheath platform moves the first and second outer sheath and the outer sheath spring along the hollow body of the handle;
a set of trigger mechanism controls comprising an inner core button configured to control movement of the inner core platform joined to the first and second inner cores, an outer sheath button configured to control movement of the outer sheath platform joined to the first and second outer sheaths, and a trigger release button configured to release the inner core spring and outer sheath spring;
wherein actuation of the trigger release button causes the outer sheath platform to eject after the inner core platform, the outer sheath platform causing the outer sheaths of the first and second needles to cut tissue as they extend over the first and second inner cores and to deposit the cut tissue in the tissue notches of the first and second needles.

2. The multiple needle biopsy instrument of claim 1 wherein the first and second needles are detachably joined to an adaptable needle mount for supporting multiple needle configurations.

3. The multiple needle biopsy instrument of claim 2 wherein the adaptable needle mount supports a third needle having a third outer sheath and a third inner core, wherein the third inner core is formed with a third tissue notch and the third outer sheath is formed coaxially around the third inner core.

4. The multiple needle biopsy instrument of claim 1 further comprising an inner core locking mechanism and an outer sheath locking mechanism.

5. The multiple needle biopsy instrument of claim 1 wherein the first needle and the second needle are formed with different gauge sizes.

6. The multiple needle biopsy instrument of claim 5 wherein the first and second needle are formed approximately 0.8 to 1 cm apart.

7. The multiple needle biopsy instrument of claim 1 wherein the inner core spring and outer sheath spring have coils with different tension strengths.

8. The multiple needle biopsy instrument of claim 1 wherein the trigger release button sequentially disengages the lock of the inner core spring before disengaging the lock of the outer sheath spring such that the inner core is ejected before the outer sheath.

9. The multiple needle biopsy instrument of claim 8 wherein the inner core platform is located at the distal end of the handle, and the trigger release button disengages the lock of the inner core spring by forcing a flange of the locking mechanism into the inner core spring's interior.

10. A system for collecting tissue from a targeted destination comprising:
a multiple needle biopsy instrument having:
a handle formed with a hollow body,
an adaptable needle mount that supports multiple needle configurations including at least a first and second needle, wherein the first and second needles each comprise an outer sheath and an inner core having a tissue notch, and the outer sheath is formed coaxially around the inner core,
an inner core platform joined to the inner cores, and coupled to an inner core spring, wherein the inner core platform moves the inner cores and the inner core spring along the hollow body of the handle,
an outer sheath platform joined to the outer sheaths, and coupled to an outer sheath spring, wherein the outer sheath platform moves the outer sheaths and the outer sheath spring along the hollow body of the handle, and
a set of trigger mechanism controls comprising an inner core button configured to control movement of the inner core platform, an outer sheath button configured to control movement of the outer sheath platform, and a trigger release button configured to release the inner core spring and outer sheath spring,
wherein actuation of the trigger release button causes the outer sheath platform to eject after the inner core platform, the outer sheath platform causing the outer sheaths of the first and second needles to cut tissue as they extend over the inner core and to deposit the cut tissue in the tissue notches of the first and second needles; and
a guide having:
a first surface for supporting a probe configured to guide the multiple needle biopsy instrument to a targeted destination, the first surface having one or more wings for securing the probe,
a needle entry barrel formed at the distal end of the guide, configured to receive the first needle and second needle,
a needle exit barrel formed at the proximal end of the guide, configured to discharge the first needle and second needle
a needle channel extending from the needle entry barrel to the needle exit barrel,
wherein the guide is configured to direct the first needle and second needle to the targeted destination.

11. The system of claim 10 wherein the needle entry barrel, needle channel, and needle exit barrel can be formed to match the configuration of the first needle and second needle on the multiple needle biopsy instrument.

12. The multiple needle biopsy instrument of claim 10 wherein the size of the needle entry barrel, needle channel, and needle exit barrel can be formed with a size to match the first needle and second needle.

13. The multiple needle biopsy instrument of claim 1 wherein the first surface is formed to have a texture or pattern that improves the grip of the guide and the probe.

14. The multiple needle biopsy instrument of claim 1 wherein the first needle and second needle are constructed of surgical steel and the handle and guide are constructed from medical grade plastic.

15. A method for collecting tissue from a targeted destination, the method comprising
providing a multiple needle biopsy instrument having:
a handle formed with a hollow body,
an adaptable needle mount for supporting multiple needle configurations of at least a first and second needle, wherein the first and second needles each comprise an outer sheath and an inner core having a tissue notch, and the outer sheath is formed coaxially around the inner core,
an inner core platform joined to the inner cores, and coupled to an inner core spring, wherein the inner core platform moves the inner cores and the inner core spring along the hollow body of the handle,
an outer sheath platform joined to the outer sheaths, and coupled to an outer sheath spring, wherein the outer sheath platform moves the outer sheaths and the outer sheath spring along the hollow body of the handle, and
a set of trigger mechanism controls comprising an inner core button configured to control movement of the inner core platform, an outer sheath button configured to control movement of the outer sheath platform, and a trigger release button configured to release the inner core spring and outer sheath spring;
providing a guide having:
a first surface for supporting a probe configured to guide the multiple needle biopsy instrument to a targeted destination, the first surface having one or more wings for securing the probe,
a needle entry barrel formed at the distal end of the guide, configured to receive the first needle and second needle,
a needle exit barrel formed at the proximal end of the guide, configured to discharge the first needle and second needle,
a needle channel extending from the needle entry barrel to the needle exit barrel, wherein the guide is configured to direct the first needle and second needle to the targeted destination; and
actuating the trigger release button causing the outer sheath platform to eject after the inner core platform, the outer sheaths of the first and second needles cutting tissue as they extend over the inner cores and depositing the cut tissue in the tissue notches of the first and second needles.
